(19) United States
(12) Patent Application Publication
(10) Pub. No.: US 2024/0394176 A1
(43) Pub. Date: Nov. 28, 2024

(54) Chatbot Evaluation System and Method

(71) Applicant: RevelAi Inc., Durham, NC (US)

(72) Inventors: Christian Pean, Durham, NC (US); Hadi Javeed, Saugus, MA (US)

(21) Appl. No.: 18/674,119

(22) Filed: May 24, 2024

Related U.S. Application Data
(60) Provisional application No. 63/503,969, filed on May 24, 2023.

(51) Int. Cl. G06F 11/36; G06F 8/65; H04L 51/02

(52) U.S. Cl. CPC G06F 11/3692; G06F 8/65; G06F 11/3688; H04L 51/02

(57) ABSTRACT

A computer-implemented method, computer program product and computing system for: providing evaluation content to a target chatbot, wherein the evaluation content includes a plurality of inquiries and a plurality of anticipated responses; processing the plurality of inquiries on the target chatbot; receiving a plurality of generated responses from the target chatbot in response to the plurality of inquiries; and comparing the plurality of generated responses received from the target chatbot to the plurality of anticipated responses included within the evaluation content.

### RELATED APPLICATION(S)

This application claims the benefit of U.S. Provisional Application No. 63/503,969, filed on 24 May 2023, the entire contents of which are herein incorporated by reference.

### TECHNICAL FIELD

This disclosure relates to evaluation systems and, more particularly, to chatbot evaluation systems.

### BACKGROUND

The history of chatbots dates back to the 1960s, beginning with ELIZA in 1966, developed by Joseph Weizenbaum at MIT. ELIZA simulated a Rogerian psychotherapist using simple pattern matching and substitution to give the illusion of understanding. In 1972, psychiatrist Kenneth Colby created PARRY, which simulated a person with paranoid schizophrenia and incorporated a model of mental processes. Moving into the 1980s and 1990s, rule-based systems emerged with chatbots like Jabberwacky (1988), developed by Rollo Carpenter, which aimed to simulate natural human conversation. Dr. Sbaitso (1992) was another early example, created by Creative Labs to demonstrate sound card capabilities.

In the 1990s and 2000s, chatbots began to see more business applications with the advent of the internet. A.L.I.C.E. (Artificial Linguistic Internet Computer Entity), developed by Richard Wallace in 1995, used heuristic pattern matching and won the Loebner Prize multiple times. SmarterChild (2001), available on AOL Instant Messenger and MSN Messenger, provided conversational interaction and information retrieval services. The integration of machine learning and AI in the 2000s brought significant advancements. Siri, Apple's virtual assistant introduced in 2011, marked the beginning of mainstream voice-activated chatbots using natural language processing. Google Now (2012) and Microsoft's Cortana (2014) followed, offering predictive search results and personal assistance.

The advent of deep learning and conversational AI in the 2010s revolutionized chatbots. Amazon's Alexa (2014) and Google Assistant (2016) demonstrated advanced conversational capabilities. OpenAI's ChatGPT, introduced in 2020, leveraged the GPT-3 model to provide highly sophisticated interactions and a wide range of applications. Despite these advancements, chatbots have faced accuracy problems throughout their history. Early chatbots like ELIZA and PARRY relied on pattern matching, limiting their ability to understand context or generate meaningful responses beyond predefined templates. Rule-based systems struggled with scalability and maintenance, as comprehensive language coverage was labor-intensive and often incomplete.

With the introduction of statistical models and machine learning, chatbots became highly data-dependent. Their accuracy hinged on the quality and diversity of training data, often inheriting biases present in the data, leading to biased or unfair responses. These models also struggled with maintaining context over long conversations, resulting in disjointed interactions. Deep learning and natural language processing improved context management but still faced challenges. Advanced models like GPT-3 require substantial computational resources, making them expensive and resource-intensive. They can also produce plausible yet incorrect or misleading information due to pattern reliance rather than true understanding. Handling nuanced language, including sarcasm, idioms, and cultural references, remains difficult.

Current challenges for chatbots include ethical concerns, ensuring the ethical use and avoidance of harmful outputs, and addressing privacy issues. The interpretability of deep learning models remains problematic, as they often function as “black boxes,” making it hard to understand their response mechanisms. Maintaining user trust, especially when errors occur, is crucial for widespread adoption. Despite these challenges, ongoing advancements in AI and natural language processing continue to enhance the accuracy and reliability of chatbots, expanding their potential across various domains.

### Summary of Disclosure

In one implementation, a computer-implemented method is executed on a computing device and includes: providing evaluation content to a target chatbot, wherein the evaluation content includes a plurality of inquiries and a plurality of anticipated responses; processing the plurality of inquiries on the target chatbot; receiving a plurality of generated responses from the target chatbot in response to the plurality of inquiries; and comparing the plurality of generated responses received from the target chatbot to the plurality of anticipated responses included within the evaluation content.

One or more of the following features may be included. The evaluation content nay include one or more of: a CSV (Comma Separated File) file; a JSON (JavaScript Object Notation) file; an XML (extensible Markup Language) file; a TSV (Tab-Separated Values) file; a PSV (Pipe-Separated Values) file; and a SSV (Space-Separated Values) file. The target chatbot may include one or more of: a rule-based chatbot; an AI-based chatbot; a hybrid chatbot; a conversational chatbot; a contextual chatbot; a voice-activated chatbot; a service/action-based chatbot; a social media chatbot; a messaging platform chatbot; and an enterprise chatbot. Comparing the plurality of generated responses received from the target chatbot to the plurality of anticipated responses included within the evaluation content may include: determining the accuracy of the target chatbot by comparing the plurality of generated responses received from the target chatbot to the plurality of anticipated responses included within the evaluation content. One or more algorithms/models associated with the target chatbot may be revised based, at least in part, upon a determined accuracy of the target chatbot. Comparing the plurality of generated responses received from the target chatbot to the plurality of anticipated responses included within the evaluation content may include: determining if the target chatbot is hallucinating by comparing the plurality of generated responses received from the target chatbot to the plurality of anticipated responses included within the evaluation content. One or more algorithms/models associated with the target chatbot may be revised based, at least in part, upon an hallucination status of the target chatbot. A report concerning the quality and/or accuracy of the generated responses received from the target chatbot may be generated. A chatbot to be evaluated for accuracy may be identified, thus defining the target chatbot. The evaluation content may be defined.

In another implementation, a computer program product resides on a computer readable medium and has a plurality of instructions stored on it. When executed by a processor, the instructions cause the processor to perform operations including: providing evaluation content to a target chatbot, wherein the evaluation content includes a plurality of inquiries and a plurality of anticipated responses; processing the plurality of inquiries on the target chatbot; receiving a plurality of generated responses from the target chatbot in response to the plurality of inquiries; and comparing the plurality of generated responses received from the target chatbot to the plurality of anticipated responses included within the evaluation content.

One or more of the following features may be included. The evaluation content nay include one or more of: a CSV (Comma Separated File) file; a JSON (JavaScript Object Notation) file; an XML (extensible Markup Language) file; a TSV (Tab-Separated Values) file; a PSV (Pipe-Separated Values) file; and a SSV (Space-Separated Values) file. The target chatbot may include one or more of: a rule-based chatbot; an AI-based chatbot; a hybrid chatbot; a conversational chatbot; a contextual chatbot; a voice-activated chatbot; a service/action-based chatbot; a social media chatbot; a messaging platform chatbot; and an enterprise chatbot. Comparing the plurality of generated responses received from the target chatbot to the plurality of anticipated responses included within the evaluation content may include: determining the accuracy of the target chatbot by comparing the plurality of generated responses received from the target chatbot to the plurality of anticipated responses included within the evaluation content. One or more algorithms/models associated with the target chatbot may be revised based, at least in part, upon a determined accuracy of the target chatbot. Comparing the plurality of generated responses received from the target chatbot to the plurality of anticipated responses included within the evaluation content may include: determining if the target chatbot is hallucinating by comparing the plurality of generated responses received from the target chatbot to the plurality of anticipated responses included within the evaluation content. One or more algorithms/models associated with the target chatbot may be revised based, at least in part, upon an hallucination status of the target chatbot. A report concerning the quality and/or accuracy of the generated responses received from the target chatbot may be generated. A chatbot to be evaluated for accuracy may be identified, thus defining the target chatbot. The evaluation content may be defined.

In another implementation, a computing system includes a processor and a memory system configured to perform operations including: providing evaluation content to a target chatbot, wherein the evaluation content includes a plurality of inquiries and a plurality of anticipated responses; processing the plurality of inquiries on the target chatbot; receiving a plurality of generated responses from the target chatbot in response to the plurality of inquiries; and comparing the plurality of generated responses received from the target chatbot to the plurality of anticipated responses included within the evaluation content.

One or more of the following features may be included. The evaluation content nay include one or more of: a CSV (Comma Separated File) file; a JSON (JavaScript Object Notation) file; an XML (extensible Markup Language) file; a TSV (Tab-Separated Values) file; a PSV (Pipe-Separated Values) file; and a SSV (Space-Separated Values) file. The target chatbot may include one or more of: a rule-based chatbot; an AI-based chatbot; a hybrid chatbot; a conversational chatbot; a contextual chatbot; a voice-activated chatbot; a service/action-based chatbot; a social media chatbot; a messaging platform chatbot; and an enterprise chatbot. Comparing the plurality of generated responses received from the target chatbot to the plurality of anticipated responses included within the evaluation content may include: determining the accuracy of the target chatbot by comparing the plurality of generated responses received from the target chatbot to the plurality of anticipated responses included within the evaluation content. One or more algorithms/models associated with the target chatbot may be revised based, at least in part, upon a determined accuracy of the target chatbot. Comparing the plurality of generated responses received from the target chatbot to the plurality of anticipated responses included within the evaluation content may include: determining if the target chatbot is hallucinating by comparing the plurality of generated responses received from the target chatbot to the plurality of anticipated responses included within the evaluation content. One or more algorithms/models associated with the target chatbot may be revised based, at least in part, upon an hallucination status of the target chatbot. A report concerning the quality and/or accuracy of the generated responses received from the target chatbot may be generated. A chatbot to be evaluated for accuracy may be identified, thus defining the target chatbot. The evaluation content may be defined.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a chatbot evaluation process in accordance with various embodiments of the present disclosure;

FIG. 2 is a flow chart of one implementation of the chatbot evaluation process of FIG. 1 in accordance with various embodiments of the present disclosure; and

FIG. 3 is a diagrammatic view of a computer system and the chatbot evaluation process of FIG. 1 coupled to a distributed computing network in accordance with various embodiments of the present disclosure.

Like reference symbols in the various drawings indicate like elements.

### DETAILED DESCRIPTION OF THE PREFERRED EMBODIMENTS

As will be discussed in greater detail below, implementations of the present disclosure may provide evaluation content to a target chatbot (wherein the evaluation content includes a plurality of inquiries and a plurality of anticipated responses); provide the plurality of inquiries to the target chatbot; receive a plurality of generated responses from the target chatbot in response to the plurality of inquiries; and compare the plurality of generated responses received from the target chatbot to the plurality of anticipated responses included within the evaluation content.

The details of one or more implementations are set forth in the accompanying drawings and the description below. Other features and advantages will become apparent from the description, the drawings, and the claims.

### Chatbot Evaluation Process:

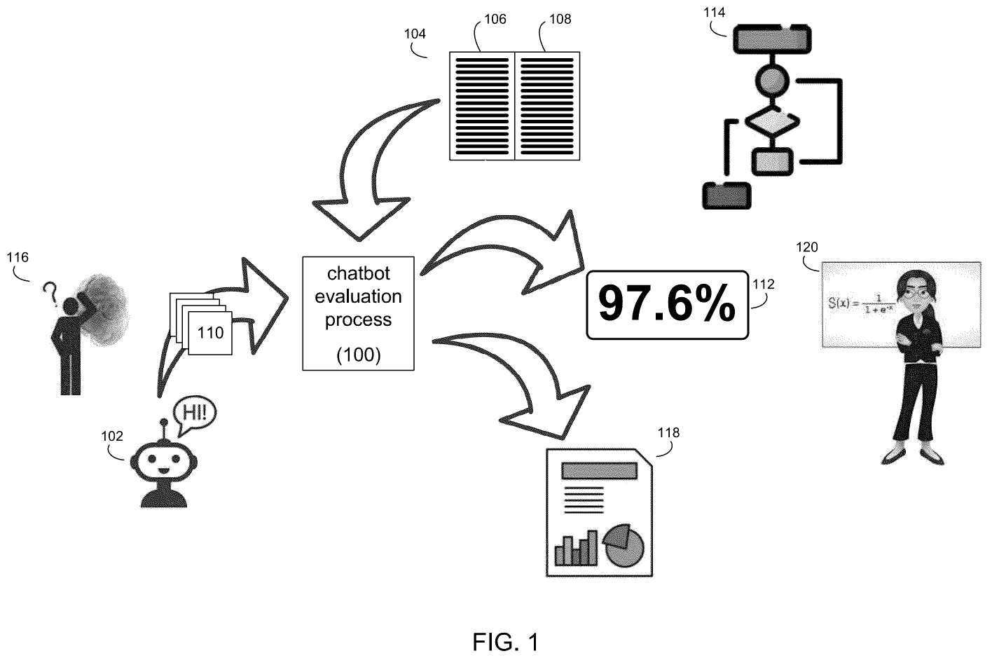

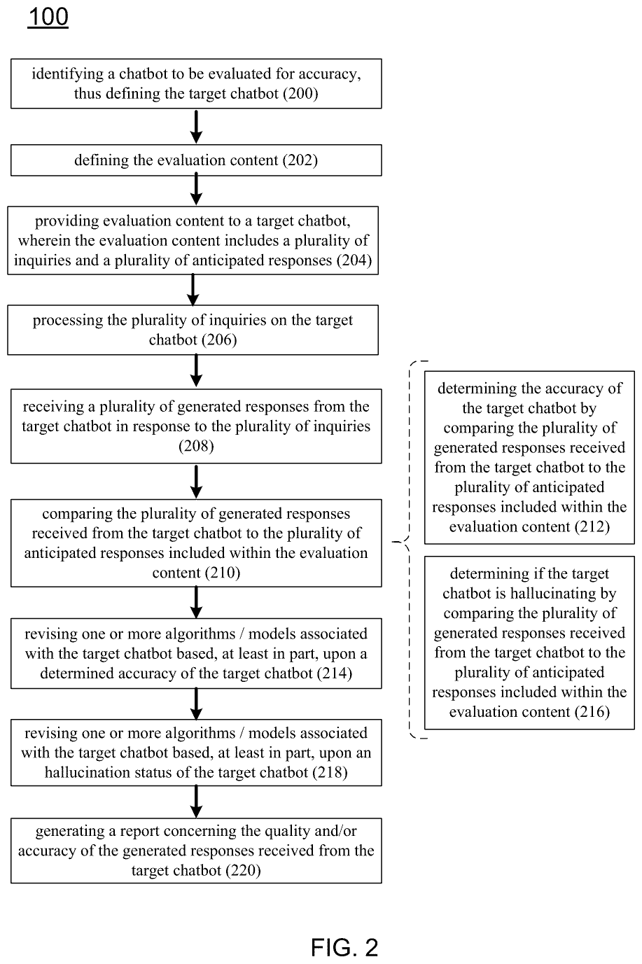

Referring to FIGS. 1-2, chatbot evaluation process 100 may identify 200 a chatbot to be evaluated for accuracy, thus defining the target chatbot (e.g., target chatbot 102).

A chatbot (e.g., target chatbot) is a software application designed to simulate human conversation through text or voice interactions. Leveraging various forms of artificial intelligence (AI), including natural language processing (NLP) and machine learning, chatbots understand and respond to user inputs in a way that mimics human communication. Chatbots perform a wide range of tasks, from answering simple queries and providing information to executing complex commands and facilitating personalized interactions. Chatbots can communicate via text-based interfaces such as websites, messaging apps, and SMS, or through voice-based interfaces like virtual assistants and smart speakers. Chatbots automate routine tasks and interactions, saving time and resources by handling repetitive queries and processes without human intervention. Advanced chatbots may use AI and NLP to understand context, interpret user intent, and generate appropriate responses, making interactions more natural and intuitive. Integration with various platforms and services, including customer support systems, e-commerce sites, social media platforms, and enterprise applications, may be a key feature of chatbots.

Chatbots may come in several types & varieties.

A Rule-Based Chatbot: Rule-based chatbots operate based on predefined rules and scripts, following set patterns of interactions and providing responses based on keywords or specific commands from users. They are commonly used for FAQs, simple customer service interactions, and basic information retrieval. These chatbots are easy to develop and deploy, offering predictable behavior but limited flexibility, making them unsuitable for handling complex queries or understanding context.

An AI-Based Chatbot: AI-based chatbots utilize artificial intelligence, machine learning, and natural language processing (NLP) to understand and respond to user inputs. Capable of learning from interactions and improving over time, they are ideal for complex customer service tasks, personalized recommendations, and interactive conversational interfaces. Although highly flexible and adept at handling complex queries, they are more complex and costly to develop, requiring substantial training data and ongoing maintenance.

A Hybrid Chatbot: Hybrid chatbots combine rule-based and AI-based approaches, using rules for simple tasks and queries while escalating to AI capabilities for more complex interactions. They are used in comprehensive customer service systems, e-commerce support, and multi-purpose applications. This approach offers a balance between simplicity and complexity, making it cost-effective and scalable, but it involves development complexity and requires careful integration of both methods.

A Conversational Chatbot: Conversational chatbots are designed to simulate human-like conversation, leveraging advanced NLP and machine learning to engage in meaningful dialogues. They are used in virtual assistants like Siri and Alexa, customer engagement, and mental health support. These chatbots provide high engagement and can handle a wide range of topics, resulting in more natural interactions. However, they pose challenges in development and training, with potential risks of generating inappropriate or biased responses.

A Contextual Chatbot: Contextual chatbots maintain context over multiple interactions and sessions, remembering past conversations to inform responses. They are used in personalized customer service, complex task management, and long-term user engagement. With their high level of personalization and improved user experience, they are effective in handling complex and multi-step queries. However, developing these chatbots requires sophisticated AI and memory management, raising data privacy concerns.

A Voice-Activated Chatbot: Voice-activated chatbots interact with users through voice commands and responses, often integrated with smart devices. Commonly seen in virtual assistants like Google Assistant and Amazon Alexa, home automation, and accessibility support, they offer hands-free interaction and intuitive use, making them accessible to a wide range of users. Challenges include speech recognition accuracy in noisy environments and privacy concerns with always-listening devices.

A Service/Action-based Chatbot: Service/action-based chatbots perform specific actions based on user commands, such as booking tickets, ordering food, or setting reminders. They are widely used in e-commerce, travel booking, and task management, providing high utility through direct and purposeful interactions that enhance efficiency. These chatbots, however, are limited to predefined actions and lack conversational flexibility.

A Social Media Chatbot: Social media chatbots are integrated into platforms like Facebook Messenger, Twitter, and Instagram to interact with users, often for customer support or marketing purposes. They have a broad reach and are easy to implement within social media, making them effective for marketing and engagement. Nonetheless, they depend on the platform and can be perceived as intrusive if not managed properly.

A Messaging Platform Chatbot: Messaging platform chatbots operate within apps such as WhatsApp, Slack, or Microsoft Teams to facilitate communication and provide services. They are used in team collaboration, customer support, and personal assistance, offering seamless integration with user workflows and enhancing communication efficiency due to their broad user base. However, they must adhere to messaging app guidelines and limitations.

An Enterprise Chatbot: Enterprise chatbots are designed for internal business use to streamline operations, provide employee support, and enhance productivity. They find applications in HR services, IT support, employee onboarding, and knowledge management, improving internal communication and boosting productivity through accessible support. Development and integration with enterprise systems can be complex, and potential security concerns must be addressed.

The applications of chatbots (e.g., target chatbot) are vast and varied. In customer support, they may provide 24/7 assistance, may answer FAQs, and resolve common issues without human intervention. In e-commerce, chatbots may assist with product recommendations, order tracking, and processing transactions. In healthcare, they may offer preliminary diagnosis, appointment scheduling, and patient follow-up. In finance, chatbots may manage account inquiries, process transactions, and provide financial advice. In education, they may tutor, answer student queries, and provide educational resources. In entertainment, they may engage users with games, stories, and interactive content. In human resources, they may streamline recruitment processes, onboarding, and employee support. Chatbots are becoming increasingly sophisticated, enabling businesses to enhance customer engagement, improve efficiency, and deliver personalized experiences. As AI and NLP technologies continue to evolve, the capabilities and applications of chatbots are expected to expand further.

Chatbot evaluation process 100 may define 202 the evaluation content (e.g., evaluation content 104). The evaluation content (e.g., evaluation content 104) may include one or more of:
A CSV (Comma Separated Values) file: A CSV file is a plain text file that uses commas to separate values. Each line in the file represents a single record, and each record consists of fields separated by commas;
A JSON (JavaScript Object Notation) file: A JSON file is a lightweight data-interchange format that is easy for humans to read and write and easy for machines to parse and generate. It uses key-value pairs and arrays to represent data structures;
An XML (extensible Markup Language) file: An XML file is a markup language that defines a set of rules for encoding documents in a format that is both human-readable and machine-readable. It uses tags to define elements and a hierarchical structure;
A TSV (Tab-Separated Values) file: A TSV file is similar to a CSV file, but it uses tabs to separate values instead of commas. Each line in the file represents a record, and fields within a record are separated by tab characters;
A PSV (Pipe-Separated Values) file: A PSV file uses the pipe character (|) to separate values. Like CSV and TSV files, each line represents a record, and fields are separated by pipes; and
A SSV (Space-Separated Values) file: An SSV file uses spaces to separate values. Each line represents a record, and fields are separated by spaces.

The evaluation content (e.g., evaluation content 104) may include a plurality of inquiries (e.g., plurality of inquiries 106) and a plurality of anticipated responses (e.g., plurality of anticipated responses 108).

Evaluating chatbots may involve creating a comprehensive set of test inquiries (e.g., plurality of inquiries 106) and anticipated responses (e.g., plurality of anticipated responses 108) to ensure the chatbot performs effectively and meets user expectations. This evaluation content (e.g., evaluation content 104) may be critical for assessing the chatbot's accuracy, reliability, and overall user experience. Inquiries (e.g., plurality of inquiries 106) are the test inputs provided to the chatbot to evaluate its responses. These inquiries (e.g., plurality of inquiries 106) should cover a wide range of scenarios and complexity levels to thoroughly assess the chatbot's capabilities. Types of inquiries (e.g., plurality of inquiries 106) include basic queries, such as simple and straightforward questions to test the chatbot's ability to handle common requests (e.g., “What is the weather today?”); complex queries, which are more intricate questions requiring the chatbot to process multiple pieces of information or perform calculations (e.g., “Can you find the nearest Italian restaurant and book a table for two at 7 P M?”); contextual queries that depend on information provided in previous interactions (e.g., first query: “What's the status of my order?” followed by: “Can you update the delivery address?”); ambiguous queries that are intentionally vague to test the chatbot's ability to request clarification or handle uncertainty (e.g., “Tell me more about that”); conversational queries designed to evaluate the chatbot's conversational skills (e.g., “What do you think about the latest news?”); and edge cases, which are uncommon questions to test the chatbot's robustness and error handling (e.g., “What happens if I enter an invalid order number?”).

Anticipated responses (e.g., plurality of anticipated responses 108) are the expected outputs from the chatbot for each inquiry (e.g., chosen from the plurality of inquiries 106), serving as benchmarks to evaluate the chatbot's performance. These responses (e.g., plurality of anticipated responses 108) should be accurate, providing correct and relevant information based on the inquiry (e.g., chosen from the plurality of inquiries 106), clear and easy to understand, context-aware for contextual queries, and maintaining a polite and appropriate tone. For error handling, anticipated responses (e.g., plurality of anticipated responses 108) should gracefully manage situations, providing useful information or next steps (e.g., “It seems the order number is invalid. Please check and try again”). In addition to inquiries (e.g., plurality of inquiries 106) and anticipated responses (e.g., plurality of anticipated responses 108), metrics may be defined to objectively evaluate the chatbot's performance. Common evaluation metrics may include response accuracy (i.e., the percentage of responses that match the anticipated responses), response time (i.e., the average time the chatbot takes to respond to inquiries), user satisfaction (i.e., feedback from users regarding their satisfaction with the chatbot's responses and overall interaction), error rate (i.e., the frequency of incorrect or failed responses), context retention (i.e., the chatbot's ability to retain and use context correctly over multiple interactions), and conversational flow (i.e., the chatbot's ability to maintain a natural and engaging conversation).

Implementing a structured testing methodology may ensure comprehensive evaluation and consistent results. The steps in the testing methodology may include designing a diverse set of inquiries covering different types and complexity levels, executing tests by interacting with the chatbot using the test inquiries (e.g., plurality of inquiries 106) and recording its responses, comparing the chatbot's responses to the anticipated responses (e.g., plurality of anticipated responses 108), analyzing the results using the defined evaluation metrics to identify strengths and areas for improvement, and iterating to refine and improve the chatbot based on the evaluation results, repeating the testing process as needed. By carefully designing and executing the evaluation content, developers can ensure that the chatbot meets performance standards and provides a positive user experience. This thorough testing process helps in identifying potential issues and enhancing the chatbot's capabilities before deployment.

Chatbot evaluation process 100 may provide 204 evaluation content (e.g., evaluation content 104) to the target chatbot (e.g., target chatbot 102), As discussed above, the evaluation content (e.g., evaluation content 104) may include a plurality of inquiries (e.g., plurality of inquiries 106) and a plurality of anticipated responses (e.g., plurality of anticipated responses 108).

Chatbot evaluation process 100 may then process 206 the plurality of inquiries (e.g., plurality of inquiries 106) on the target chatbot (e.g., target chatbot 102).

Generally speaking, the plurality of inquiries (e.g., plurality of inquiries 106) that are processed 206 on the target chatbot (e.g., target chatbot 102) are the typical types of questions that would be provided to the target chatbot (e.g., target chatbot 102) during the normal operation of the same. For example, if the target chatbot (e.g., target chatbot 102) is to be used in the personal assistant space, such plurality of inquiries (e.g., plurality of inquiries 106) may include questions like “What is the weather today?” and “When do we switch to daylight savings time?” And if the target chatbot (e.g., target chatbot 102) is to be used in the customer service space, such plurality of inquiries (e.g., plurality of inquiries 106) may include questions like “What is the warranty period for an X27A dehumidifier?” and “When do you think the Ultra Electric Toothbrush will be back in stock?” And if the target chatbot (e.g., target chatbot 102) is to be used in the post-acute care space, such plurality of inquiries (e.g., plurality of inquiries 106) may include questions like “How often do I need to take my blood pressure medicine?” and “Is it normal for my incision to be inflamed and swollen?”

Chatbot evaluation process 100 may receive 208 a plurality of generated responses (e.g., plurality of generated responses 110) from the target chatbot (e.g., target chatbot 102) in response to the plurality of inquiries (e.g., plurality of inquiries 106).

As could be imagined, the plurality of generated responses (e.g., plurality of generated responses 110) that are received 208 from the target chatbot (e.g., target chatbot 102) should correspond to (or be related to) the questions that were processed 206 by the target chatbot (e.g., target chatbot 102). For example: if the question processed 206 (within plurality of inquiries 106) was “What is the weather today?”, the generated response received 208 should concern a weather forecast; if the question processed 206 (within plurality of inquiries 106) was “When do we switch to daylight savings time?”, the generated response received 208 should concern a date “; if the question processed 206 (within plurality of inquiries 106) was “What is the warranty period for an X27A dehumidifier?”, the generated response received 208 should concern a period of time; if the question processed 206 (within plurality of inquiries 106) was “When do you think the Ultra Electric Toothbrush will be back in stock?”, the generated response received 208 should concern a period of time or a date; if the question processed 206 (within plurality of inquiries 106) was “How often do I need to take my blood pressure medicine?”, the generated response received 208 should concern a time period/frequency; and if the question processed 206 (within plurality of inquiries 106) was “Is it normal for my incision to be inflamed and swollen?”, the generated response received 208 should concern a decision.

Chatbot evaluation process 100 may compare 210 the plurality of generated responses (e.g., plurality of generated responses 110) received from the target chatbot (e.g., target chatbot 102) to the plurality of anticipated responses (e.g., plurality of anticipated responses 108) included within the evaluation content (e.g., evaluation content 104).

Continuing with the above-stated examples:

If the question processed 206 (within plurality of inquiries 106) was “What is the weather today?”, the corresponding anticipated response (e.g., within plurality of anticipated responses 108) may be something like “70 degrees and sunny”. If the response received 208 from the target chatbot (e.g., target chatbot 102) was “68 degrees and rainy”, chatbot evaluation process 100 may compare 210 the generated response received 208 and the anticipated response (e.g., defined within plurality of anticipated responses 108) and may consider this to be an acceptable answer as it is in the proper form (assuming that this answer is technically accurate). However, if the generated response received 208 from the target chatbot (e.g., target chatbot 102) was “−42 degrees and rainy”, chatbot evaluation process 100 may compare 210 this generated response received 208 and the anticipated response (e.g., defined within plurality of anticipated responses 108) and may consider this to be an unacceptable answer as it is technically inaccurate (i.e., it cannot be −42 degrees and raining) even though it is in the proper form (i.e., a weather forecast).

If the question processed 206 (within plurality of inquiries 106) was “When do we switch to daylight savings time?”, the corresponding anticipated response (e.g., within plurality of anticipated responses 108) may be something like “March 10^{th}”. If the response received 208 from the target chatbot (e.g., target chatbot 102) was “March 16^{th}”, chatbot evaluation process 100 may compare 210 the generated response received 208 and the anticipated response (e.g., defined within plurality of anticipated responses 108) and may consider this to be an acceptable answer as it is in the proper form (assuming that this answer is technically accurate). However, if the generated response received 208 from the target chatbot (e.g., target chatbot 102) was “Green”, chatbot evaluation process 100 may compare 210 this generated response received 208 and the anticipated response (e.g., defined within plurality of anticipated responses 108) and may consider this to be an unacceptable answer as it is not in the proper form (i.e., green is not a date) and is technically inaccurate (i.e., green is not a date).

If the question processed 206 (within plurality of inquiries 106) was “What is the warranty period for an X27A dehumidifier?”, the corresponding anticipated response (e.g., within plurality of anticipated responses 108) may be something like “3 years from the date of purchase”. If the response received 208 from the target chatbot (e.g., target chatbot 102) was “12 months from the date of purchase”, chatbot evaluation process 100 may compare 210 the generated response received 208 and the anticipated response (e.g., defined within plurality of anticipated responses 108) and may consider this to be an acceptable answer as it is in the proper form (assuming that this answer is technically accurate). However, if the generated response received 208 from the target chatbot (e.g., target chatbot 102) was “157 years”, chatbot evaluation process 100 may compare 210 this generated response received 208 and the anticipated response (e.g., defined within plurality of anticipated responses 108) and may consider this to be an unacceptable answer as it is technically inaccurate (i.e., the warranty is not 157 years) even though it is in the proper form (i.e., a quantity of time).

If the question processed 206 (within plurality of inquiries 106) was “When do you think the Ultra Electric Toothbrush will be back in stock?”, the corresponding anticipated response (e.g., within plurality of anticipated responses 108) may be something like “it should be back in stock the end of this month”, If the response received 208 from the target chatbot (e.g., target chatbot 102) was “within the next four weeks”, chatbot evaluation process 100 may compare 210 the generated response received 208 and the anticipated response (e.g., defined within plurality of anticipated responses 108) and may consider this to be an acceptable answer as it is in the proper form (assuming that this answer is technically accurate). However, if the generated response received 208 from the target chatbot (e.g., target chatbot 102) was “How dare you ask that”, chatbot evaluation process 100 may compare 210 this generated response received 208 and the anticipated response (e.g., defined within plurality of anticipated responses 108) and may consider this to be an unacceptable answer as it is not in the proper form (i.e., the answer is rude and noninformative) and is technically inaccurate (i.e., no answer was provided).

If the question processed 206 (within plurality of inquiries 106) was “How often do I need to take my blood pressure medicine?”, the corresponding anticipated response (e.g., within plurality of anticipated responses 108) may be something like “once per day . . . in the morning . . . after eating”. If the response received 208 from the target chatbot (e.g., target chatbot 102) was “everyday at noon with food”, chatbot evaluation process 100 may compare 210 the generated response received 208 and the anticipated response (e.g., defined within plurality of anticipated responses 108) and may consider this to be an acceptable answer as it is in the proper form (assuming that this answer is technically accurate). However, if the generated response received 208 from the target chatbot (e.g., target chatbot 102) was “I am not sure . . . sorry”, chatbot evaluation process 100 may compare 210 this generated response received 208 and the anticipated response (e.g., defined within plurality of anticipated responses 108) and may consider this to be an unacceptable answer as it is not in the proper form (i.e., an answer was not provided) and is technically inaccurate (i.e., an answer was not provided).

If the question processed 206 (within plurality of inquiries 106) was “Is it normal for my incision to be inflamed and swollen?”, the corresponding anticipated response (e.g., within plurality of anticipated responses 108) may be something like “Possibly, as your surgery was just 2 days ago. Keep an eye on it and let's chat tomorrow”. If the response received 208 from the target chatbot (e.g., target chatbot 102) was “Not a concern, but let's keep an eye on it”, chatbot evaluation process 100 may compare 210 the generated response received 208 and the anticipated response (e.g., defined within plurality of anticipated responses 108) and may consider this to be an acceptable answer as it is in the proper form (assuming that this answer is technically accurate). However, if the generated response received 208 from the target chatbot (e.g., target chatbot 102) was “You may have Trocitrus Syndrome”, chatbot evaluation process 100 may compare 210 this generated response received 208 and the anticipated response (e.g., defined within plurality of anticipated responses 108) and may consider this to be an unacceptable answer as it is not in the proper form (i.e., this is a hallucination) and is technically inaccurate (i.e., there is no such thing as Trocitrus Syndrome).

When comparing 210 the plurality of generated responses (e.g., plurality of generated responses 110) received from the target chatbot (e.g., target chatbot 102) to the plurality of anticipated responses (e.g., plurality of anticipated responses 108) included within the evaluation content (e.g., evaluation content 104), chatbot evaluation process 100 may determine 212 the accuracy of the target chatbot (e.g., target chatbot 102) by comparing the plurality of generated responses (e.g., plurality of generated responses 110) received from the target chatbot (e.g., target chatbot 102) to the plurality of anticipated responses (e.g., plurality of anticipated responses 108) included within the evaluation content (e.g., evaluation content 104). This may result in chatbot evaluation process 100 generating an accuracy score (e.g., accuracy score 112) that identifies the perceived accuracy of the target chatbot (e.g., target chatbot 102) based upon e.g., the ratio of acceptable vs. unacceptable answers.

Chatbot evaluation process 100 may revise 214 one or more algorithms/models (e.g., algorithms/models 114) associated with the target chatbot (e.g., target chatbot 102) based, at least in part, upon a determined accuracy of the target chatbot (e.g., target chatbot 102). For example, if the accuracy of the target chatbot (e.g., target chatbot 102) is perceived to be too low (e.g., less than 98%), the algorithms/models (e.g., algorithms/models 114) associated with the target chatbot (e.g., target chatbot 102) may be revised to enhance accuracy.

Chatbots (e.g., target chatbot 102) may leverage algorithms and AI models (e.g., algorithms/models 114) to interact with users in a human-like manner, providing responses and performing tasks based on user input. At the core of chatbots is Natural Language Processing (NLP), which helps chatbots understand and interpret user inputs, whether text or speech. NLP involves breaking down sentences into words (tokenization), identifying parts of speech (part-of-speech tagging), recognizing entities like names and dates (named entity recognition), and analyzing sentiment to gauge the user's emotions or intentions.

Machine learning models may play a crucial role in chatbots (e.g., target chatbot 102). These models are trained on large datasets to recognize patterns and make predictions. Supervised learning may involve training models on labeled data, while unsupervised learning lets models identify patterns without explicit instructions. Reinforcement learning allows models to learn from feedback, improving performance over time.

Deep learning, particularly neural networks, may model complex patterns in data. Advanced models, like those using transformers (e.g., GPT-4), may handle nuanced language tasks and understand context, enabling coherent conversations over multiple exchanges. Dialogue management may be another essential aspect, involving intent recognition (determining what the user wants to achieve) and slot filling (extracting necessary details to complete a task). Response generation may be rule-based, template-based, or AI-generated, depending on the chatbot's design.

Context management may ensure that chatbots maintain the conversation context, providing relevant and coherent responses. User profiling may store user preferences and past interactions, allowing for personalized responses and an improved user experience. Reinforcement learning may create a feedback loop where chatbots learn from user interactions, refining their models with each interaction based on positive and negative feedback.

Further and when comparing 210 the plurality of generated responses (e.g., plurality of generated responses 110) received from the target chatbot (e.g., target chatbot 102) to the plurality of anticipated responses (e.g., plurality of anticipated responses 108) included within the evaluation content (e.g., evaluation content 104), chatbot evaluation process 100 may determine 216 if the target chatbot (e.g., target chatbot 102) is hallucinating by comparing the plurality of generated responses (e.g., plurality of generated responses 110) received from the target chatbot (e.g., target chatbot 102) to the plurality of anticipated responses (e.g., plurality of anticipated responses 108) included within the evaluation content (e.g., evaluation content 104). This may result in chatbot evaluation process 100 identifying and mitigating one or more hallucinations (e.g., hallucination 116).

Chatbot evaluation process 100 may revise 218 one or more algorithms/models (e.g., algorithms/models 114) associated with the target chatbot (e.g., target chatbot 102) based, at least in part, upon a hallucination status of the target chatbot (e.g., target chatbot 102).

Chatbot hallucinations (e.g., hallucination 116) may refer to instances where a chatbot generates responses that are factually incorrect, nonsensical, or completely fabricated. This phenomenon may occur when the language model creates content that appears plausible but has no basis in reality or relevant data. Hallucinations may manifest in several ways, including incorrect information, invented facts, or responses that do not align with the context of the conversation.

Several factors may cause chatbot hallucinations. One major factor is training data limitations. If the training data includes inaccuracies or lacks coverage of certain topics, the model may generate incorrect responses based on these gaps or errors. Another cause may be model overconfidence; language models can sometimes produce confident-sounding responses even when they are guessing or have no relevant information, leading to plausible-sounding but incorrect answers. Additionally, chatbots might misunderstand the context or intent of the user's query, resulting in responses that do not fit the conversation. Lastly, some models incorporate a degree of randomness to generate varied and natural-sounding responses, which can occasionally result in hallucinations.

Examples of chatbot hallucinations may include providing incorrect information, such as incorrectly claiming that the capital of Australia is Sydney instead of Canberra. They might also fabricate facts, like inventing a statistic that “30% of people prefer green apples over red,” without any supporting data. Another example is producing nonsensical responses, such as answering a question about the weather with a response about the stock market.

To mitigate chatbot hallucinations, several strategies may be employed. One is improving the training data by ensuring it is comprehensive, accurate, and up-to-date, reducing the likelihood of hallucinations. Model calibration techniques may help prevent the model from presenting guesses as facts by adjusting its confidence levels. Enhancing the model's contextual awareness to better maintain and understand the conversation context may also reduce irrelevant or nonsensical responses. Lastly, implementing systems where human reviewers can oversee and correct chatbot responses (human-in-the-loop) can help ensure accuracy and relevance. By addressing these issues, the accuracy and reliability of chatbot responses can be significantly improved.

Chatbot evaluation process 100 may generate 220 a report (e.g., report 118) concerning the quality and/or accuracy of the generated responses (e.g., plurality of generated responses 110) received from the target chatbot (e.g., target chatbot 102). Report 118 may be made available to a data scientist (e.g., data scientist 120) associated with the target chatbot (e.g., target chatbot 102) so that the data scientist (e.g., data scientist 120) may review the report (e.g., report 118) to determine is the accuracy of the target chatbot (e.g., target chatbot 102) is sufficient and/or the algorithms/models (e.g., algorithms/models 114) associated with the target chatbot (e.g., target chatbot 102) should be revised.

### System Overview

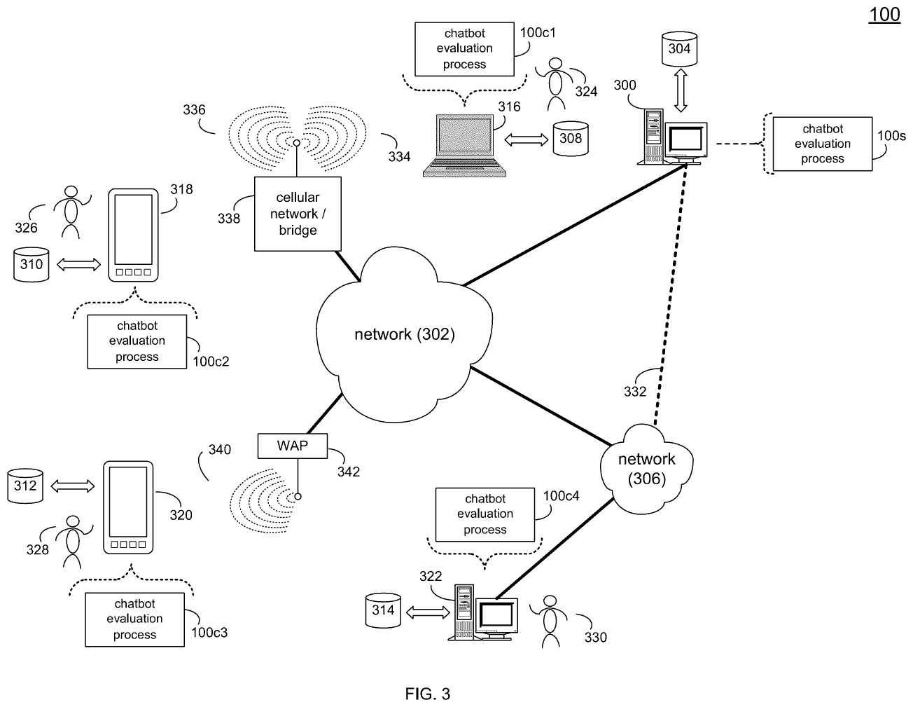

Referring to FIG. 3, there is shown chatbot evaluation process 100. Chatbot evaluation process 100 may be implemented as a server-side process, a client-side process, or a hybrid server-side/client-side process. For example, chatbot evaluation process 100 may be implemented as a purely server-side process via chatbot evaluation process 100s. Alternatively, chatbot evaluation process 100 may be implemented as a purely client-side process via one or more of chatbot evaluation process 100cl, chatbot evaluation process 100c2, chatbot evaluation process 100c3, and chatbot evaluation process 100c4. Alternatively still, chatbot evaluation process 100 may be implemented as a hybrid server-side/client-side process via chatbot evaluation process 100s in combination with one or more of chatbot evaluation process 100c1, chatbot evaluation process 100c2, chatbot evaluation process 100c3, and chatbot evaluation process 100c4.

Accordingly, chatbot evaluation process 100 as used in this disclosure may include any combination of chatbot evaluation process 100s, chatbot evaluation process 100c1, chatbot evaluation process 100c2, chatbot evaluation process 100c3, and chatbot evaluation process 100c4.

Chatbot evaluation process 100s may be a server application and may reside on and may be executed by computing device 300, which may be connected to network 302 (e.g., the Internet or a local area network). Examples of computing device 300 may include, but are not limited to: a personal computer, a server computer, a series of server computers, a mini computer, a mainframe computer, a smartphone, or a cloud-based computing platform.

The instruction sets and subroutines of chatbot evaluation process 100s, which may be stored on storage device 304 coupled to computing device 300, may be executed by one or more processors (not shown) and one or more memory architectures (not shown) included within computing device 300. Examples of storage device 304 may include but are not limited to: a hard disk drive; a RAID device; a random-access memory (RAM); a read-only memory (ROM); and all forms of flash memory storage devices.

Network 302 may be connected to one or more secondary networks (e.g., network 306), examples of which may include but are not limited to: a local area network; a wide area network; or an intranet, for example.

Examples of chatbot evaluation processes 300c1, 300c2, 300c3, 300c4 may include but are not limited to a web browser, a game console user interface, a mobile device user interface, or a specialized application (e.g., an application running on e.g., the Android™ platform, the iOS™ platform, the Windows™ platform, the Linux™ platform or the UNIX™ platform). The instruction sets and subroutines of chatbot evaluation processes 300c1, 300c2, 300c3, 300c4, which may be stored on storage devices 308, 310, 312, 314 (respectively) coupled to client electronic devices 316, 318, 320, 322 (respectively), may be executed by one or more processors (not shown) and one or more memory architectures (not shown) incorporated into client electronic devices 316, 318, 320, 322 (respectively). Examples of storage devices 308, 310, 312, 314 may include but are not limited to: hard disk drives; RAID devices; random access memories (RAM); read-only memories (ROM), and all forms of flash memory storage devices.

Examples of client electronic devices 316, 318, 320, 322 may include, but are not limited to a personal digital assistant (not shown), a tablet computer (not shown), laptop computer 316, smart phone 318, smart phone 320, personal computer 322, a notebook computer (not shown), a server computer (not shown), a gaming console (not shown), and a dedicated network device (not shown). Client electronic devices 316, 318, 320, 322 may each execute an operating system, examples of which may include but are not limited to Microsoft Windows™, Android™, iOS™, Linux™, or a custom operating system.

Users 324, 326, 328, 330 may access chatbot evaluation process 10 directly through network 302 or through secondary network 306. Further, chatbot evaluation process 10 may be connected to network 302 through secondary network 306, as illustrated with link line 332.

The various client electronic devices (e.g., client electronic devices 316, 318, 320, 322) may be directly or indirectly coupled to network 302 (or network 306). For example, laptop computer 316 and smart phone 318 are shown wirelessly coupled to network 302 via wireless communication channels 334, 336 (respectively) established between laptop computer 316, smart phone 318 (respectively) and cellular network/bridge 338, which is shown directly coupled to network 302.

Further, smart phone 320 is shown wirelessly coupled to network 302 via wireless communication channel 340 established between smart phone 320 and wireless access point (i.e., WAP) 342, which is shown directly coupled to network 302. Additionally, personal computer 322 is shown directly coupled to network 306 via a hardwired network connection.

WAP 342 may be, for example, an IEEE 802.11a, 802.11b, 802.11 g, 802.11n, Wi-Fi, and/or Bluetooth device that is capable of establishing wireless communication channel 340 between smart phone 320 and WAP 342. As is known in the art, IEEE 802.11x specifications may use Ethernet protocol and carrier sense multiple access with collision avoidance (i.e., CSMA/CA) for path sharing. As is known in the art, Bluetooth is a telecommunications industry specification that allows e.g., mobile phones, computers, and personal digital assistants to be interconnected using a short-range wireless connection.

### General:

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be used. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet.

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, not at all, or in any combination with any other flowcharts depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
providing evaluation content to a target chatbot, wherein the evaluation content includes a plurality of inquiries and a plurality of anticipated responses;
processing the plurality of inquiries on the target chatbot;
receiving a plurality of generated responses from the target chatbot in response to the plurality of inquiries; and
comparing the plurality of generated responses received from the target chatbot to the plurality of anticipated responses included within the evaluation content.

2. The computer-implemented method of claim 1 wherein the evaluation content includes one or more of:
a CSV (Comma Separated File) file;
a JSON (JavaScript Object Notation) file;
an XML (extensible Markup Language) file;
a TSV (Tab-Separated Values) file;
a PSV (Pipe-Separated Values) file; and
a SSV (Space-Separated Values) file.

3. The computer-implemented method of claim 1 wherein the target chatbot includes one or more of:
a rule-based chatbot;
an AI-based chatbot;
a hybrid chatbot;
a conversational chatbot;
a contextual chatbot;
a voice-activated chatbot;
a service/action-based chatbot;
a social media chatbot;
a messaging platform chatbot; and
an enterprise chatbot.

4. The computer-implemented method of claim 1 wherein comparing the plurality of generated responses received from the target chatbot to the plurality of anticipated responses included within the evaluation content includes:
determining the accuracy of the target chatbot by comparing the plurality of generated responses received from the target chatbot to the plurality of anticipated responses included within the evaluation content.

5. The computer-implemented method of claim 4 further comprising:
revising one or more algorithms/models associated with the target chatbot based, at least in part, upon a determined accuracy of the target chatbot.

6. The computer-implemented method of claim 1 wherein comparing the plurality of generated responses received from the target chatbot to the plurality of anticipated responses included within the evaluation content includes:
determining if the target chatbot is hallucinating by comparing the plurality of generated responses received from the target chatbot to the plurality of anticipated responses included within the evaluation content.

7. The computer-implemented method of claim 6 further comprising:
revising one or more algorithms/models associated with the target chatbot based, at least in part, upon an hallucination status of the target chatbot.

8. The computer-implemented method of claim 1 further comprising:
generating a report concerning the quality and/or accuracy of the generated responses received from the target chatbot.

9. The computer-implemented method of claim 1 further comprising:
identifying a chatbot to be evaluated for accuracy, thus defining the target chatbot.

10. The computer-implemented method of claim 1 further comprising:
defining the evaluation content.

11. A computer program product residing on a computer readable medium having a plurality of instructions stored thereon which, when executed by a processor, cause the processor to perform operations comprising:
providing evaluation content to a target chatbot, wherein the evaluation content includes a plurality of inquiries and a plurality of anticipated responses;
processing the plurality of inquiries on the target chatbot;
receiving a plurality of generated responses from the target chatbot in response to the plurality of inquiries; and
comparing the plurality of generated responses received from the target chatbot to the plurality of anticipated responses included within the evaluation content.

12. The computer program product of claim 11 wherein the evaluation content includes one or more of:
a CSV (Comma Separated File) file;
a JSON (JavaScript Object Notation) file;
an XML (extensible Markup Language) file;
a TSV (Tab-Separated Values) file;
a PSV (Pipe-Separated Values) file; and
a SSV (Space-Separated Values) file.

13. The computer program product of claim 11 wherein the target chatbot includes one or more of:
a rule-based chatbot;
an AI-based chatbot;
a hybrid chatbot;
a conversational chatbot;
a contextual chatbot;
a voice-activated chatbot;
a service/action-based chatbot;
a social media chatbot;
a messaging platform chatbot; and
an enterprise chatbot.

14. The computer program product of claim 11 wherein comparing the plurality of generated responses received from the target chatbot to the plurality of anticipated responses included within the evaluation content includes:
determining the accuracy of the target chatbot by comparing the plurality of generated responses received from the target chatbot to the plurality of anticipated responses included within the evaluation content.

15. The computer program product of claim 14 further comprising:
revising one or more algorithms/models associated with the target chatbot based, at least in part, upon a determined accuracy of the target chatbot.

16. The computer program product of claim 11 wherein comparing the plurality of generated responses received from the target chatbot to the plurality of anticipated responses included within the evaluation content includes:
determining if the target chatbot is hallucinating by comparing the plurality of generated responses received from the target chatbot to the plurality of anticipated responses included within the evaluation content.

17. The computer program product of claim 16 further comprising:
revising one or more algorithms/models associated with the target chatbot based, at least in part, upon an hallucination status of the target chatbot.

18. The computer program product of claim 11 further comprising:
generating a report concerning the quality and/or accuracy of the generated responses received from the target chatbot.

19. The computer program product of claim 11 further comprising:
identifying a chatbot to be evaluated for accuracy, thus defining the target chatbot.

20. The computer program product of claim 11 further comprising:
defining the evaluation content.

21. A computing system including a processor and memory configured to perform operations comprising:
providing evaluation content to a target chatbot, wherein the evaluation content includes a plurality of inquiries and a plurality of anticipated responses;
processing the plurality of inquiries on the target chatbot;
receiving a plurality of generated responses from the target chatbot in response to the plurality of inquiries; and
comparing the plurality of generated responses received from the target chatbot to the plurality of anticipated responses included within the evaluation content.

22. The computing system of claim 21 wherein the evaluation content includes one or more of:
a CSV (Comma Separated File) file;
a JSON (JavaScript Object Notation) file;
an XML (extensible Markup Language) file;
a TSV (Tab-Separated Values) file;
a PSV (Pipe-Separated Values) file; and
a SSV (Space-Separated Values) file.

23. The computing system of claim 21 wherein the target chatbot includes one or more of:
a rule-based chatbot;
an AI-based chatbot;
a hybrid chatbot;
a conversational chatbot;
a contextual chatbot;
a voice-activated chatbot;
a service/action-based chatbot;
a social media chatbot;
a messaging platform chatbot; and
an enterprise chatbot.

24. The computing system of claim 21 wherein comparing the plurality of generated responses received from the target chatbot to the plurality of anticipated responses included within the evaluation content includes:
determining the accuracy of the target chatbot by comparing the plurality of generated responses received from the target chatbot to the plurality of anticipated responses included within the evaluation content.

25. The computing system of claim 24 further comprising:
revising one or more algorithms/models associated with the target chatbot based, at least in part, upon a determined accuracy of the target chatbot.

26. The computing system of claim 21 wherein comparing the plurality of generated responses received from the target chatbot to the plurality of anticipated responses included within the evaluation content includes:
determining if the target chatbot is hallucinating by comparing the plurality of generated responses received from the target chatbot to the plurality of anticipated responses included within the evaluation content.

27. The computing system of claim 26 further comprising:
revising one or more algorithms/models associated with the target chatbot based, at least in part, upon an hallucination status of the target chatbot.

28. The computing system of claim 21 further comprising:
generating a report concerning the quality and/or accuracy of the generated responses received from the target chatbot.

29. The computing system of claim 21 further comprising:
identifying a chatbot to be evaluated for accuracy, thus defining the target chatbot.

30. The computing system of claim 21 further comprising:
defining the evaluation content.
